(19) United States
(12) Patent Application Publication
(10) Pub. No.: US 2026/0160687 A1
(43) Pub. Date: Jun. 11, 2026

(54) SENSOR ELECTRODE FOR HALOGEN DETERMINATION AND METHOD FOR MANUFACTURING A SENSOR ELECTRODE

(71) Applicant: Endress+Hauser Conducta GmbH+Co. KG, Gerlingen (DE)

(72) Inventors: Ralf Bernhard, Stuttgart (DE); Andreas Bayer, Gerlingen (DE); Andreas Müller, Ostfildern (DE)

(21) Appl. No.: 19/409,117

(22) Filed: Dec. 4, 2025

(30) Foreign Application Priority Data
Dec. 11, 2024 (DE) 10 2024 137 273.7

(51) Int. Cl. G01N 21/64; G01N 21/65; G01N 33/00

(52) U.S. Cl. CPC G01N 21/645; G01N 21/65; G01N 33/0052; G01N 2021/6471; G01N 2201/0633; G01N 2201/0636

(57) ABSTRACT

An optical system for a measuring device for optically measuring a measured variable of a medium comprises a first signal path in which a filter designed as a bandpass interference filter is inserted at an inclination angle different from zero degrees, which divides the first signal path into a unidirectional transmission path and a bidirectional path used to transmit excitation radiation and to receive measurement radiation. The inclination angle is dimensioned such that the inclined filter transmits signal components lying within its passband of transmission radiation supplied to the transmission path as excitation radiation, and reflects signal components lying outside its passband of measurement radiation impinging thereon via the bidirectional path and resulting from interactions of the transmitted excitation radiation with the medium, and makes them available as useful radiation.

### CROSS-REFERENCE TO RELATED APPLICATION

The present application is related to and claims the priority benefit of German Patent Application No. 10 2024 137 273.7, filed on Dec. 11, 2024, the entire contents of which are incorporated herein by reference.

### TECHNICAL FIELD

The present disclosure relates to an optical system for a measuring device for optically measuring at least one measured variable of a medium, which variable can be acquired by measurement technology on the basis of useful radiation that results from a wavelength-changing interaction and is contained in measurement radiation resulting from interactions of excitation radiation with the medium, to a mount for an optical system, and to a measuring device for the optical measurement of at least one measured variable having an optical system.

### BACKGROUND

Measuring devices for the optical measurement of at least one measured variable are used in a wide variety of applications. These applications include for example applications in the chemical industry, the oil and gas industries, the food industry, water and wastewater systems such as sewage treatment plants, as well as applications in measurement and automation technology.

Among other things, measurement methods are used in which excitation radiation is sent into a medium, measurement radiation resulting from interactions between the excitation radiation and the medium is received, and the measurement variables are determined on the basis of useful radiation contained in the measurement radiation and which results from a wavelength-changing interaction, such as fluorescence or Raman scattering, of the excitation radiation with the medium.

For this purpose, measuring devices with optical systems for example are used which comprise a bidirectional transmission path via which the excitation radiation is sent into the medium and the measurement radiation resulting from interactions of the excitation radiation with the medium is received. This offers the advantage that only one process interface is required that is in contact with the medium during measurement operation, via which interface the excitation radiation is sent and the measurement radiation is received. A disadvantage, however, is that the measurement radiation received via the bidirectional transmission path usually contains a portion of the excitation radiation reflected back in the medium without wavelength changes, which portion must be separated from the wavelength-changed useful radiation contained in the measurement radiation.

For this signal separation, optical systems can be used that comprise a bandpass filter that narrowly limits the wavelength range of the transmitted excitation light and a beam splitter that is tuned to the wavelength ranges to be separated. Beam splitters suitable for this purpose are usually custom-made and are highly precisely tailored to the wavelength ranges to be separated, and are correspondingly expensive.

U.S. Pat. No. 6,907,149 B2 describes an optical system for a Raman probe or a fluorescence sensor, which comprises a first signal path running along a first optical axis and a second signal path running along a second optical axis running parallel to the first optical axis. Transmission radiation is fed into the first signal path by a radiation source and is collimated by a lens inserted into the first signal path on the input side. In addition, a bandpass filter is inserted into the first signal path following the lens in the transmission direction, which filter eliminates interference radiation and limits the wavelength range of the transmitted excitation radiation to a specified transmission wavelength range.

Furthermore, the optical system comprises a reflector inserted into the end of the first signal path, which reflects the excitation radiation transmitted by the bandpass filter in the direction of a dielectric edge filter inserted into the second signal path. The edge filter serves as a beam splitter, by means of which the second signal path is divided into a bidirectional transmission path for transmitting the excitation radiation and for receiving the measurement radiation, and a unidirectional transmission path for outputting the useful radiation. For this purpose, the edge filter is oriented at an inclination angle to the second optical axis such that it reflects excitation radiation impinging thereon in a transmission direction running along the bidirectional transmission path, and transmits the useful radiation contained in the measurement radiation impinging thereon via the bidirectional transmission path into the unidirectional transmission path.

In principle, the inclination angle can be 45°, for example. According to U.S. Pat. No. 6,907,149 B2, a greater insensitivity of the edge filter to the polarization of the radiation impinging thereon is achieved by orienting the edge filter at an inclination angle of less than or equal to 20° to the second optical axis.

However, in order to achieve the most complete possible separation of the excitation radiation contained in the measurement radiation and reflected by the medium without a change in wavelength, the optical system described in U.S. Pat. No. 6,907,149 B2 requires a highly precise tuning of the edge filter used in the second signal path to the passband of the bandpass filter inserted into the first signal path.

For this purpose, a bandpass filter with the narrowest possible passband can be used in the first signal path and/or a high-quality edge filter with the narrowest possible transition range between the permeable spectral range and the opaque spectral range of the edge filter can be used in the second signal path. However, correspondingly high-quality edge filters are usually expensive optical components.

In addition, there is the problem that the optical properties of the edge filter change depending on the angles of incidence at which the excitation radiation and the measurement radiation impinge thereon. The angle of incidence of the excitation radiation depends not only on the orientation of the reflector but also on the inclination angle of the edge reflector. The angle of incidence of the measuring beam depends on the inclination angle of the edge reflector. Accordingly, the optical system described in U.S. Pat. No. 6,907,149 B2 requires highly precise positioning and orienting of the reflector and the edge filter. This increases the manufacturing costs and the sensitivity of the optical system to vibrations.

According to an optional embodiment, a notch filter is used in the unidirectional transmission path of the optical system described in U.S. 6,907,149 B2, with which filter any portions of the reflected excitation radiation still contained in the portion of the measurement radiation transmitted by the edge filter are eliminated. However, this increases the number of optical components in the optical system, which leads to a corresponding increase in manufacturing costs.

### SUMMARY

It is an object of the present disclosure to provide an optical system with which the most complete separation possible of the excitation radiation contained in the measurement radiation and reflected by the medium from the useful radiation can be achieved in a manner that is as cost-effective as possible and is easier to implement in terms of manufacturing technology.

For this purpose, the present disclosure comprises an optical system for a measuring device for optically measuring at least one measured variable of a medium, which variable can be acquired by measurement technology on the basis of useful radiation that results from a wavelength-changing interaction and is contained in measurement radiation resulting from interactions of excitation radiation with the medium, wherein the optical system:
comprises a first signal path running along a first optical axis and a filter inserted into the first signal path, wherein the filter:
divides the first signal path into a unidirectional transmission path and a bidirectional path used to transmit excitation radiation and to receive measurement radiation,
is designed as a bandpass interference filter, and
is inclined relative to the first optical axis such that an inclination angle different from zero degrees is enclosed between a normal vector to the inclined filter and the first optical axis, wherein the inclination angle is dimensioned such that the inclined filter:
transmits signal components, lying within a passband of the inclined filter, of the transmission radiation supplied to the transmission path as excitation radiation, and
reflects signal components, lying outside the passband of the inclined filter, of measurement radiation impinging on the filter via the bidirectional path and resulting from interactions of the transmitted excitation radiation with the medium, and makes said signal components available as useful radiation.

The optical system offers the advantage that the filter inserted into the first signal path performs not only the function of a bandpass filter that eliminates interference signals and limits the wavelength range of the transmitted excitation radiation but also the function of a beam splitter that separates the reflected excitation radiation contained in the measurement radiation from the useful radiation.

This dual function offers the advantage that the bandpass filter formed by the filter and the beam splitter formed by the filter are perfectly matched to each other not only in terms of their spectral transmission behavior but also in terms of their position and orientation. This offers the advantage that the filter only reflects signal components of the measurement radiation that lie outside the wavelength range of the transmitted excitation radiation, which are then available as useful radiation without the need for a highly precise orientation and positioning of the filter.

This offers the additional advantage that a bandpass interference filter, which is available inexpensively as a standard component, can easily be used as the filter. In addition, the dual function of the filter offers the advantage of reducing the number of optical components required. Both lead to a corresponding reduction in production costs.

In a development, the inclination angle is greater than or equal to 5° and the inclination angle is less than or equal to 45°, less than or equal to 35° or less than or equal to 20°.

In a further development, the inclination angle is dimensioned such that a central wavelength of the inclined filter corresponds to a target wavelength specified for the excitation radiation.

In embodiments:
a collimation device is inserted into the transmission path, which collimation device is designed to collimate transmission radiation fed into the transmission path, wherein the collimation device comprises at least one optical component and/or a lens, and/or
an optical device is inserted into the bidirectional path, which optical device is designed to focus excitation radiation emerging from the optical system via the bidirectional path onto a region lying outside the optical system and to collimate measurement radiation entering the bidirectional path from the region, wherein the optical device comprises at least one optical component, a lens, and/or an objective.

A preferred development consists in the optical system comprising a second signal path running along a second optical axis or a second optical axis running parallel to the first optical axis, wherein
a reflector is inserted into the second signal path at the end, and
the reflector is arranged relative to the inclined filter and is inclined relative to the second optical axis in such a way that useful radiation reflected by the inclined filter impinges on the reflector and is reflected in a direction parallel to the second optical axis.

In embodiments of the preferred development:
a focusing device is inserted into the second signal path, which focusing device is designed to focus useful radiation emerging from the optical system via the second signal path onto a region located inside or outside the optical system, wherein the focusing device comprises at least one optical component and/or a lens, and/or
an additional filter is inserted into the second signal path, which filter limits a wavelength range of useful radiation transmitted through the additional filter to a measuring wavelength range specified depending on the measured variable to be measured.

Furthermore, the present disclosure comprises a mount for an optical system, which system comprises at least one optical component arranged in a first signal path running along a first optical axis and at least one optical component arranged in a second signal path running along a second optical axis or along a second optical axis running parallel to the first optical axis, wherein the mount comprises two half-shells, and wherein:
each half-shell has for each optical axis a recess running parallel to the optical axis,
the half-shells can be positioned or are positioned on one another in such a way that each pair of recesses of the half-shells arranged on top of one another, formed by two opposite, adjacent recesses, forms in each case a channel whose longitudinal axis is coaxial with one of the optical axes,
each half-shell for each optical component of the optical system has a pocket adjacent to one of the recesses, into which a partial region of an outer edge of the corresponding optical component can be or is inserted, and
each pocket is arranged at a position provided for the corresponding optical component that can be inserted or is inserted therein within the optical system in an orientation specified for the optical component relative to the optical axis running through the optical component in the optical system.

A first development of the mount provides that:
the adjacent recesses of each half-shell are separated from each other by a partition arranged therebetween,
wherein the half-shells are designed such that:
the partitions of the stacked half-shells are adjacent to each other,
the partitions extend over the entire length of the recesses adjacent thereto, and/or
in each of the partitions of the half-shells a recess is provided, which is designed such that the mutually adjacent recesses of the half-shells arranged on top of one another form a passage opening for a connecting path running from one of the optical axes to the other optical axis, and/or
the half-shells can be connected or are connected to one another by mutually complementary plug connector elements arranged on the end faces of the partitions of the half-shells.

A further development of the device consists in:
the adjacent recesses of each half-shell being separated from each other by a partition arranged therebetween, and
at least one plug connector element designed as a tongue or as a tongue running over the entire length of the partition being arranged on the partition of at least one of the two half-shells, and the partition of the other half-shell comprises, for each plug connector element designed as a tongue, a plug connector element complementary thereto designed as a groove.

According to a second development, the mount comprises a sleeve and is designed such that:
the half-shells arranged on top of one another can be inserted or are inserted into the sleeve, and/or
the half-shells are designed such that
the half-shells arranged on top of one another can be inserted or are inserted into the sleeve, and/or
they consist entirely, or at least in sections, of an elastically deformable material or an elastically deformable plastic and/or have a shape which enables and/or effects the clamping of the half-shells arranged on top of one another in the sleeve.

A further development of the second development consists in the half-shells having on their outer side projecting structures and/or structures designed as projections, as connecting elements or as elongated structural elements, which are designed and arranged such that, in cooperation with the sleeve surrounding the half-shells arranged on top of one another, they effect an external clamping of the optical components arranged in the half-shells arranged on top of one another.

A development of the last-mentioned development consists in
the structures of each half-shell comprising for each optical component at least one structure which projects outwards relative to a shell region of the corresponding half-shell, in which the pocket provided for the optical component is arranged on the inside, and/or
the half-shells for at least one or each optical component oriented perpendicular to one of the optical axes in the optical system each comprising at least one structure running parallel to the optical axis, and/or
the half-shells for at least one or each optical component tilted in the optical system relative to one of the optical axes by an angle specified for the corresponding component each comprising a structure extending at the angle specified for the component relative to the corresponding optical axis.

A development of the second development of the mount consists in
the half-shells having openings into each of which a shaped seal can be inserted or is inserted,
the openings of each half-shell being designed and arranged in such a way that at least two openings distributed in the circumferential direction around the outer edge adjoin the outer edge of each optical component inserted into the half-shells arranged on top of one another, each opening exposing a partial region of the outer edge of the optical component, and
the half-shells arranged on top of one another, with the optical components inserted therein and the shaped seals inserted into the openings, being insertable or being inserted into the sleeve in such a way that the shaped seals are clamped between the partial regions, adjacent thereto on the inside, of the outer edges of the optical components and the sleeve.

A further development of the mount consists in
the half-shells being elastically deformable at least in the region of the pockets such that the optical components can be or are clamped in the pockets,
the half-shells as a whole or at least in the region of the pockets consisting of an elastically deformable material or an elastically deformable plastic and/or in the region of the pockets have a shape that enables and/or effects the clamping of the optical components, and/or
the half-shells comprising mutually complementary plug connector elements by means of which the half-shells can be or are mechanically connected to one another.

Furthermore, the present disclosure comprises a measuring device for measuring at least one measured variable of a medium with an optical system according to the present disclosure, which device:
comprises a housing in which the optical system is arranged,
comprises a transmitting device with a radiation source which is designed to feed transmitted radiation generated by the radiation source into the transmission path of the optical system, and
comprises a detection device which is designed to receive useful radiation emitted by the optical system and to determine and provide at least one property of the useful radiation which is dependent on the measured variable.

According to a development, the measuring device comprises a mount according to the present disclosure.

Embodiments consist in the measuring device:
comprising a transparent process interface through which the measuring device transmits excitation radiation into the medium and receives measurement radiation resulting from interactions of the transmitted excitation radiation with the medium,
comprising an evaluation device which is connectable or is connected to the detection device and which is designed to determine and provide measurement results of the measured variable based on the property or properties of the useful radiation determined by the detection device,
being designed as a fluorescence measuring device or as a Raman spectrometric measuring device, and/or
being designed as a fluorescence measuring device in such a way that:
the radiation source is designed to generate transmitted radiation in a wavelength range matched to a fluorescent component of the medium and/or in a wavelength range of 180 nm to 1200 nm, and/or
the inclination angle is dimensioned such that the inclined filter has a central wavelength which corresponds to a desired wavelength with which a fluorescent component of the medium can be excited to fluorescence.

### BRIEF DESCRIPTION OF THE DRAWINGS

The present disclosure and its advantages will now be explained in detail using the figures in the drawing, which show several examples of embodiments. The same elements are indicated by the same reference signs in the figures.

FIG. 1 shows an optical system;

FIG. 2 shows transmission curves of a bandpass interference filter;

FIG. 3 shows a measuring device with an optical system;

FIG. 4 shows a mount for an optical system;

FIG. 5 shows a first half-shell of the mount shown in FIG. 4;

FIG. 6 shows a second half-shell of the mount shown in FIG. 4, equipped with optical components of the optical system shown in FIG. 1;

FIG. 7 shows a housing of the measuring device shown in FIG. 3, in which the optical components of the optical system are arranged in a mount; and

FIG. 8: shows a further embodiment of a mount.

### DETAILED DESCRIPTION

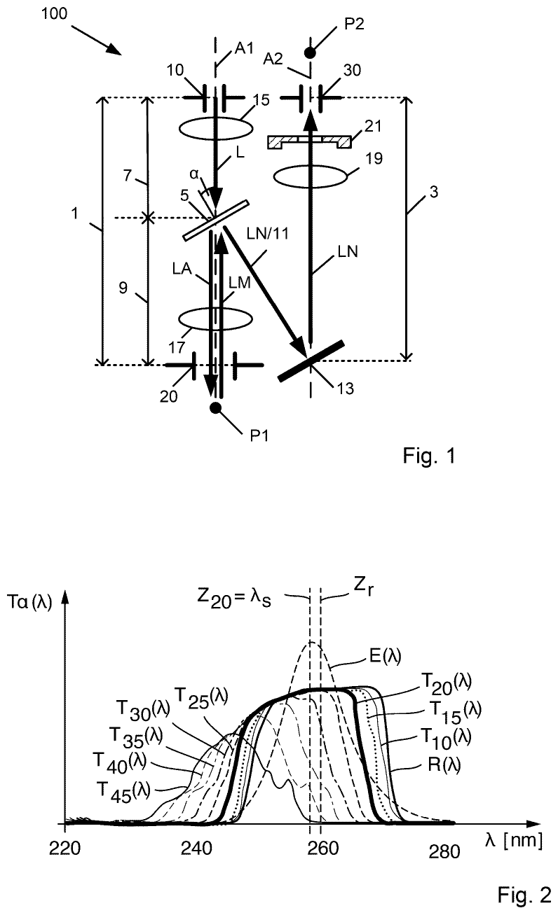

The present disclosure comprises an optical system 100 for a measuring device 200 for optically measuring at least one measured variable of a medium, which variable can be acquired by measurement technology on the basis of useful radiation that results from a wavelength-changing interaction and is contained in measurement radiation resulting from interactions of excitation radiation with the medium. An exemplary embodiment of the optical system 100 is shown in FIG. 1.

The optical system 100 is designed to emit excitation radiation LA via an interface 20 in the direction of a medium, based on transmission radiation L supplied to the optical system 100 via an input 10 of the optical system 100, to receive measurement radiation LM resulting from interactions of the excitation light LA with the medium via the interface 20, and via an output 30 of the optical system 100 to output useful radiation LN contained in the measurement radiation LM and resulting from a wavelength-changing interaction of the excitation light LA with the medium.

For this purpose, the optical system 100 comprises a first signal path 1 running along a first optical axis A1. An optional embodiment which is advantageous, inter alia with regard to a design which is as compact as possible and/or with regard to the structure of a measuring device 200 comprising the optical system 100, consists in the optical system 100, in addition to the first signal path 1, comprising a second signal path 3 which is also shown as an option in FIG. 1 and runs along a second optical axis A2. FIG. 1 shows an exemplary embodiment in which the second optical axis A2 runs parallel to the first optical axis A1. However, this is not absolutely necessary.

A filter 5 designed as a bandpass interference filter is inserted into the first signal path 1, by means of which filter the first signal path 1 is divided into a unidirectional transmission path 7 and a bidirectional path 9 used to transmit the excitation radiation LA and to receive the measurement radiation LM.

Suitable bandpass interference filters are filters available as standard components, such as those offered by the Semrock company. These standard components are usually used as a bandpass filter in such a way that the radiation to be filtered impinges on the filter in a direction parallel to the normal vector to the filter.

In contrast, the filter 5 in the optical system 100 is inclined relative to the first optical axis A1 such that an inclination angle α different from zero degrees is enclosed between a normal vector to the inclined filter 5 and the first optical axis A1. The inclination angle α is dimensioned such that the inclined filter 5 transmits signal components, lying within a passband of the inclined filter 5 dependent on the size of the inclination angle α, of transmission radiation L supplied to the transmission path 7 via the input 10 of the optical system 100, into the bidirectional path 9 as excitation radiation LA. The inclination angle α is thus also dimensioned such that the inclined filter 5 reflects signal components lying outside the passband of the inclined filter 5 of measurement radiation LM and impinging on the filter via the bidirectional path 9 and resulting from interactions of the transmitted excitation radiation LA with the medium, and makes them available as useful radiation LN.

The dimensioning of the inclination angle α is illustrated in FIG. 2 using the example of transmission curves Tα(λ) of a bandpass interference filter, which this bandpass interference filter exhibits at different inclination angles α of 10°, 15°, 20°, 25°, 30°, 40° and 45°. A bandpass interference filter from the Semrock company was used, which, with respect to radiation impinging on it parallel to its normal vector, has a transmission curve, also shown in FIG. 2 as a reference curve R(λ), with a central wavelength Zᵣ of 260 nm.

As can be seen from FIG. 2, the inclination angle α, which is different from zero degrees, causes a deviation, which deviation becomes larger with increasing size of the inclination angle α, of the spectral transmission curve Tα(λ) and of the central wavelength Z_{α} of the inclined filter 5 from the reference curve R(λ) and the central wavelength Zᵣ that the bandpass interference filter has with respect to radiation impinging on it in a direction parallel to the normal vector. As this example shows, there is a relatively large inclination angle range for bandpass interference filters in which bandpass interference filters exhibit the transmission and reflection properties stated for the inclined filter 5. Correspondingly, the inclination angle α of the inclined filter 5 is determined such that it lies within this inclination angle range.

The filter 5, which is inclined by the inclination angle α lying within this inclination angle range, offers the advantage that in the optical system 100 it assumes not only the function of a bandpass filter which eliminates interference radiation and limits the wavelength range of the transmitted excitation radiation LA but also the function of a beam splitter which separates from the useful radiation LN the excitation radiation LA that is contained in the measurement radiation LM and that re-enters the optical system 100 without any wavelength changes. The inclined filter 5 separates the excitation radiation LA reflected from the medium and, if applicable, the excitation radiation LA scattered back from the medium, without wavelength changes. In addition, the inclined filter 5 also separates any excitation radiation LA that may be reflected and/or scattered back along the optical path running from the optical system 100 to the medium without wavelength changes, such as for example excitation radiation LA that may be reflected and/or scattered back by elements that may be present in the vicinity of the optical path.

Since the inclined filter 5 is transparent in the limited passband, signal components lying within the passband of the inclined filter 5 are not reflected by measurement radiation LM impinging on it via the bidirectional path 9, but are transmitted into the transmission path 7. Since the passband at the same time also corresponds to the wavelength range of the excitation radiation LA emitted via the bidirectional path 9, this ensures that the signal component reflected at the inclined filter 5 of the measurement radiation LM impinging thereon via the bidirectional path 9 is free of excitation radiation LA returning to the bidirectional path 9 without any wavelength changes, and thus has wavelengths located unchanged within the passband, without the need for a highly precise orientation of the inclined filter 5 for this purpose.

The optical system 100 has the aforementioned advantages. Optionally, individual components and/or regions of the optical system 100 can each have different embodiments that can be used individually and/or in combination with one another.

In this respect, the signal component of the measurement radiation LM reflected at the inclined filter 5 can be output for example via a useful signal path 11 running in a straight line from the inclined filter 5 to a correspondingly positioned output of the optical system 100. Here the angle at which the useful signal path 11 runs relative to the first optical axis A1 is given by the inclination angle α of the filter 5.

FIG. 1 shows an alternative exemplary embodiment in which the optical system 100 comprises, in addition to the first signal path 1, the second signal path 3 running along the second optical axis A2. In this embodiment, a reflector 13 is inserted into the second signal path 3 at its end. This reflector 13 is arranged relative to the inclined filter 5 such that, and is inclined relative to the second optical axis A2 such that, useful radiation LN reflected by the inclined filter 5 impinges on the reflector 13 and is reflected in a direction parallel to the second optical axis A2. In conjunction with the parallel orientation of the two optical axes A1, A2, this offers the advantage that the useful radiation LN is output via the useful signal path 11 running from the inclined filter 5 to the reflector 13, the reflector 13, the second signal path 3 and the correspondingly positioned output 30 on that side of the optical system 100 at which the transmitted radiation L is also fed into the optical system 100.

An advantageous embodiment, particularly with regard to the coupling-out of the useful radiation LN reflected at the inclined filter 5, consists in the inclination angle α being greater than or equal to 5°. Here, in particular with regard to the transmittance of the inclined filter 5 in the passband decreasing with increasing size of the inclination angle α, it is advantageous if the inclination angle α is less than 45°, the inclination angle α preferably being less than or equal to 35° or even less than or equal to 20°.

Alternatively or in addition, the selection of the bandpass interference filter used as filter 5 and the dimensioning of the size of the inclination angle α are matched to one another for example in such a way that the central wavelength Zα of the inclined filter 5 corresponds to a target wavelength λs specified for the excitation radiation LA. This offers the advantage that the transmitted excitation radiation LA is narrowband or even approximately monochromatic radiation whose wavelengths lie within the limited passband of the inclined filter 5 comprising the target wavelength λs.

Alternatively or in addition to the previously described embodiments, the optical system 100 can comprise at least one further optical component. FIG. 1 shows, as an exemplary embodiment of this, a collimation device 15 inserted into the transmission path 7, which is designed to collimate the transmission radiation L fed into the transmission path 7 via an input 10 of the optical system 100. This collimation device 15 is designed for example such that it comprises at least one optical component, such as a lens.

A further embodiment also shown in FIG. 1 consists in an optical device 17 being used in the bidirectional path 9, which device is designed to focus excitation radiation LA transmitted by the optical system 100 onto a region P1 lying outside the optical system 100, such as a focal point, a focal line or a limited surface, and to collimate measurement radiation LM entering the bidirectional path 9 from the region P1. This optical device 17 is designed for example such that it comprises at least one optical component, such as an objective and/or a lens.

Alternatively or additionally, at least one further optical component can also be provided in the second signal path 3. An exemplary embodiment of this shown in FIG. 1 consists in a focusing device 19, such as a lens, being inserted into the second signal path 3 on the output side, which focusing device is designed to focus useful radiation LN emitted via the second signal path 3 onto a region P2 located inside or outside the optical system 100, such as a focal point, a focal line or a limited surface.

An exemplary embodiment also shown as an option in FIG. 1 consists in an additional filter 21 being inserted into the second signal path 3, which filter limits the wavelength range of the useful radiation LN transmitted through the additional filter 21 to a measuring wavelength range specified depending on the measured variable to be measured.

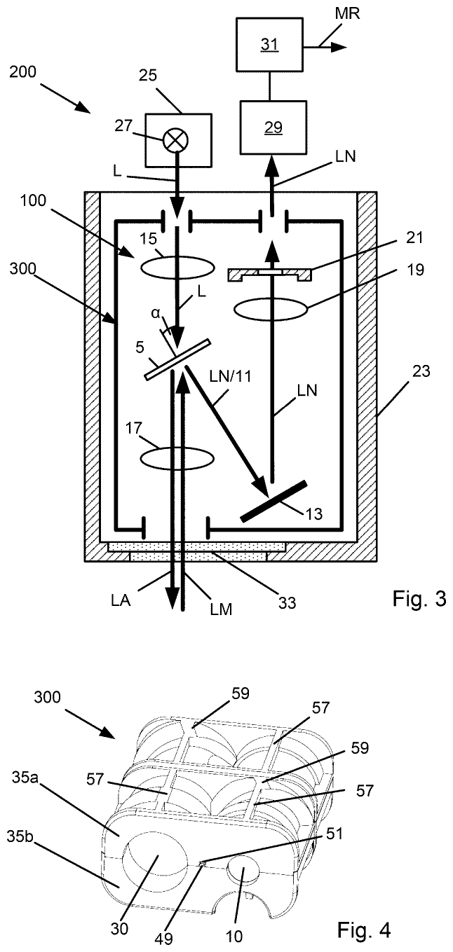

The optical system 100 described above is used, for example, in a measuring device 200 shown in FIG. 3 for measuring at least one measured variable that can be acquired by measurement technology on the basis of the useful radiation LN. The measurement device 200 comprises a housing 23 in which the optical system 100 is arranged. Furthermore, the measuring device 200 comprises a transmitting device 25 having a radiation source 27, which is designed to feed transmitted radiation L generated by the radiation source 27 into the transmission path 7 of the optical system 100, and a detection device 29, which is designed to receive the useful radiation LN emitted by the optical system 100 and to acquire, using measuring technology, and make available at least one property of the useful radiation LN that is dependent on the measured variable, such as at least one spectral intensity or an intensity spectrum of the useful radiation LN.

The measuring device 200 has the advantages already described in connection with the optical system 100. Optionally, individual components of the measuring device 200 can each have different embodiments that can be used individually and/or in combination with one another.

In this respect, the detection device 29 is designed, for example, to display the property or properties of the useful radiation LN that depend on the measured variable, to output them as detection signals and/or to make them available to an evaluation device 31 that is designed as a component of the measuring device 200 or that is connectable or connected to the detection device 29. This evaluation device 31 is designed, for example, to determine and provide measurement results MR of the measured variable on the basis of the property or properties of the useful radiation LN determined by the detection device 29.

A further embodiment shown in FIG. 3 consists in the measuring device 200 comprising a transparent process interface 33 through which the measuring device 200 transmits excitation radiation LA generated by the transmitting device 25 and the optical system 100 into the medium and receives measurement radiation LM resulting from interactions of the transmitted excitation radiation LA with the medium. For this purpose, the process interface 33 comprises for example a window inserted into the housing, through which excitation radiation LA transmitted via the bidirectional path 9 is sent into the medium and through which the measurement radiation LM enters the bidirectional path 9. Suitable for this purpose is for example a window made of glass, sapphire, quartz, plastic or another material that is transparent in the wavelength range of the excitation radiation LA and in the wavelength range of the useful radiation LN. Alternatively, however, the process interface 33 may also comprise a housing wall region of the housing that is transparent in the wavelength ranges specified above.

The transmitting device 27, the detection device 29 and/or the evaluation device 31 can be designed in different ways known from the prior art, depending on the type of wavelength-changing interaction used to determine the measured variable and/or the measured variable(s).

One embodiment variant consists in the measuring device 200 being designed as a fluorescence measuring device. In this case, the medium comprises at least one component that can be excited to fluorescence by the excitation radiation LA, and the measuring device 200 is designed for example to measure at least one measured variable of the medium that can be measured on the basis of the fluorescent light emitted by the medium, such as a concentration of the fluorescent component contained in the medium.

In this embodiment variant, the radiation source 27 is designed for example as a light source that generates light with a wavelength range matched to the fluorescent component of the medium. Depending on the type of fluorescent component, the radiation source 27 is designed for example to generate light in a wavelength range of 180 nm to 1200 nm. In this respect, the radiation source 27 comprises for example a light-emitting diode (LED), an incandescent lamp, a flash lamp, a gas discharge lamp or a laser.

In this embodiment, the central wavelength Zα, which the filter 5 tilted at the inclination angle α has, is preferably set to a desired wavelength λₛ with which the fluorescent component can be excited to fluorescence. For this purpose, the inclined filter 5 is used, for example at the inclination angle α at which the central wavelength Zα of the correspondingly inclined filter 5 is equal to the target wavelength λₛ.

The inclination angle α, which is different from zero degrees, here has the result that the central wavelength Zα of the inclined filter 5, corresponding to the target wavelength λs, deviates from the central wavelength Zᵣ which this filter 5 has with respect to radiation impinging on the filter 5 parallel to the normal vector. This is shown in FIG. 2 using the example of a target wavelength λₛ of 255 nm. In this example, e.g. a light source, such as a UV light-emitting diode, is used as the radiation source 27, the emission spectrum E of which, also shown in FIG. 2, has a pronounced maximum at the target wavelength λₛ. As can be seen from FIG. 2, the bandpass interference filter, which has the transmission behavior shown in FIG. 2 for different sizes of the inclination angle α, is used to tune to the desired wavelength λₛ of 255 nm, for example at an inclination angle α of the order of 20°, at which the central wavelength Z₂₀ of the correspondingly inclined filter 5 is equal to the target wavelength λₛ. In this case, the difference between the central wavelength Z₂₀ of 255 nm occurring at the inclination angle α of 20° of the bandpass interference filter and the central wavelength Zᵣ of 260 nm, which this bandpass interference filter has with respect to radiation impinging on it parallel to the normal vector, is 5 nm.

When the measuring device 200 is designed as a fluorescence measuring device, the detection device 29 comprises for example a measuring device, for example a photodiode, a photodiode array or a spectrometer, which receives the useful radiation LN and determines a property or properties of the useful radiation LN that are dependent on the measured variable, such as at least one spectral intensity value and/or an intensity spectrum of the useful radiation LN.

The present disclosure can also be used analogously in conjunction with optical measuring principles in which a different wavelength-changing interaction of the excitation radiation LA with the medium is used. An example of this is Raman scattering. In this respect, the measuring device 200 is designed for example as a Raman spectrometric measuring device. In this case, the radiation source 27 is preferably a monochromatic light source, such as a laser, which emits transmitted radiation L in a wavelength range suitable for exciting Raman scattering, such as transmitted radiation in the visible or near infrared range, and the detection device 29 comprises a spectrometer which determines and provides Raman spectra of the medium on the basis of the useful radiation LN. In this embodiment, the inclined filter 5 designed as a bandpass interference filter offers the advantage that the useful radiation LN can contain signal components caused by both Stokes scattering and anti-Stokes scattering.

During the manufacture and/or installation of optical systems, such as the optical system 100 shown in FIG. 1, it is necessary to arrange the optical components of the optical system in a defined position and orientation relative to one another, which is predetermined by their function, and to fix them in this arrangement. This becomes more complex the greater the number of optical components and the greater the number of optical axes along which at least one of the optical components has to be arranged. In addition, a plurality of components to be arranged along an optical axis, at least one of which has to be oriented in an orientation tilted relative to the optical axis, cannot easily be arranged on top of one another in a stack.

In this respect, the present disclosure also comprises in particular a mount 300 for an optical system, such as the optical system 100 shown in FIG. 1, which comprises a first signal path 1 running along a first optical axis A1 and a second signal path 3 running along a second optical axis A2, such as a second optical axis A2 running parallel to the first optical axis A1, in which at least one optical component is arranged in each of the first signal path 1 and the second signal path 3.

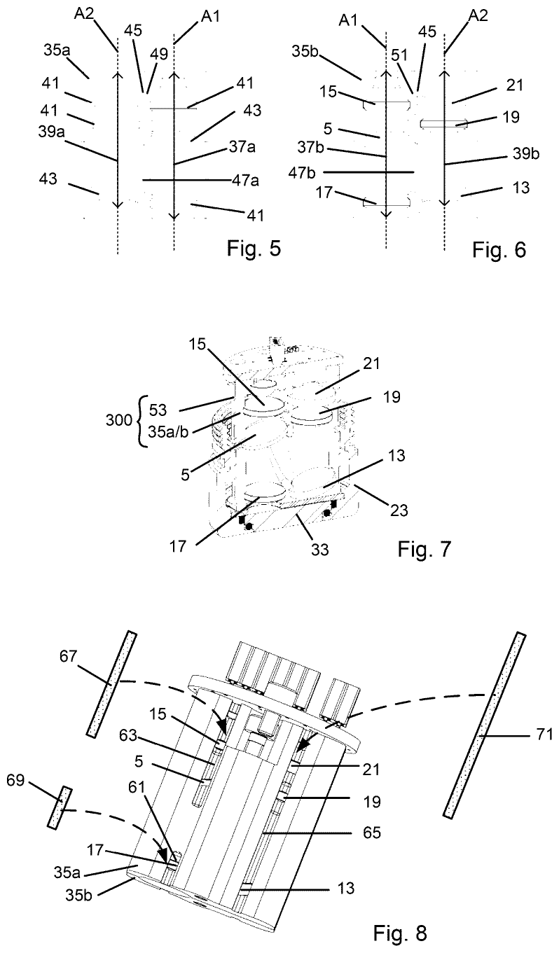

An exemplary embodiment of the mount 300 is shown in FIG. 4. The mount 300 comprises two half-shells 35a, 35b. One of the two half-shells 35a of the mount 300 shown in FIG. 4 is shown in FIG. 5. The other half-shell 35b is shown in FIG. 6.

Each of the two half-shells 35a, 35b has, for each of the optical axes A1, A2, a recess 37a, 37b, 39a, 39b running parallel to the corresponding optical axis A1, A2. Furthermore, the half-shells 35a, 35b can be positioned on one another in the manner shown in FIG. 4 such that the recesses 37a, 37b, 39a, 39b are opposite one another in pairs and the opposite recesses 37a, 37b, 39a, 39b are adjacent to one another.

As can be seen from FIG. 4 to 6, the recesses 37a, 37b, 39a, 39b are designed such that each pair of recesses formed by two of the paired opposite recesses 37a, 37b, 39a, 39b of the half-shells 35a, 35b arranged on top of one another forms a channel whose longitudinal axis is coaxial with one of the optical axes A1, A2.

In addition, each half-shell 35a, 35b for each optical component of the optical system 100 has a pocket 41, 43 adjacent to one of the recesses 37a, 37b, 39a, 39b, into which a partial region of an outer edge of the corresponding optical component can be or is inserted.

For this purpose, pockets 41, 43 are designed for example in such a way that they each have a cross-sectional geometry that corresponds to the cross-sectional geometry of the outer edge of the optical component that can be or is inserted therein. In this respect, the pockets 41, 43, depending on the design of the optical component that can be inserted or is inserted therein, are designed for example as a groove or notch open towards the adjacent recess 37a, 37b, 39a, 39b and having a corresponding cross-sectional geometry.

Independently of the embodiment in this regard, each pocket 41, 43 is arranged at a position provided for the optical component that can be inserted or is inserted therein within the optical system 100 in an orientation specified for the corresponding component within the optical system 100 relative to the optical axis A1, A2 running through the corresponding optical component in the optical system 100.

Here the optical components comprise, for example, at least one component in the optical system 100 oriented perpendicular to one of the optical axes A1, A2 and/or at least one component in the optical system 100 tilted relative to one of the optical axes A1, A2. In the present case, vertically oriented components are components that are oriented in the optical system 100 such that a normal vector to the component runs parallel to one of the optical axes A1, A2. Tilted components herein are components which are oriented in the optical system 100 such that a normal vector to the component runs at an angle to one of the optical axes A1, A2 which is different from zero degrees and which is specified for the tilted component.

In this respect, the half-shells 35a, 35 each comprise pockets 41 oriented perpendicular to the corresponding optical axis A1, A2 for each optical component oriented perpendicular to one of the optical axes A1, A2. This is illustrated in FIGS. 5 and 6 using the example of the pockets 41 provided in the half-shells 35a, 35 for the components of the collimation device 15 shown in FIG. 1, the optical device 17 shown in FIG. 1, the focusing device 19 shown in FIG. 1, and the additional filter 21 shown in FIG. 1, which pockets are oriented perpendicular to the corresponding optical axis A1, A2.

Analogously, the half-shells 35a, 35 each comprise, for each component tilted relative to one of the optical axes A1, A2, a pocket 43 which is inclined at the angle specified for the corresponding component relative to the optical axis A1, A2 running through the respective component in the optical system 100. This is shown in FIGS. 5 and 6 using the example of the pockets 43 provided in the half-shells 35a, 35 for the filter 5 inclined relative to the first optical axis A1 and the reflector 13 inclined relative to the second optical axis A2, each inclined relative to the corresponding optical axis A1, A2 at the angle provided for the optical component that can be inserted or is inserted therein.

The mount 300 offers the advantage that one of the two half-shells 35a can be equipped with the optical components in a very simple manner and the other half-shell 35a can then be arranged on the equipped half-shell 35b. FIG. 5 shows an exemplary embodiment of this in which the half-shell 35b is equipped with the components of the optical system 100 shown in FIG. 1. Accordingly, the components shown here as examples comprise the inclined filter 5, the reflector 15, and if appropriate also the optical components of the collimation device 15, the optical device 17, the focusing device 19, and/or the additional filter 21. When the other half-shell 35b is being arranged on the equipped half-shell 35a, the optical components are automatically also inserted into the pockets 41, 43 provided for this purpose in the other half-shell 35b.

The mount 300 offers the advantage that the position and orientation of the pockets 41, 43 also determine the position and orientation of the optical components inserted therein relative to one another and to the optical axes A1, A2.

A further advantage is that by arranging the second half-shell 35b on the equipped half-shell 35a, an assembly is created which is robust, easy to handle and can be inserted as a module into a housing of a measuring device, such as the housing 23 of the measuring device 200 shown in FIG. 3. Relating to this, FIG. 7 shows a sectional view of the measuring device 200 shown in FIG. 3, in which the mount 300 with the optical components of the optical system 100 shown in FIG. 1 enclosed therein is arranged in the housing 31 of the measuring device 200.

The mount 300 has the advantages mentioned above. Optionally, individual components and/or regions of the mount 300 can each have different embodiments that can be used individually and/or in combination with one another.

One embodiment consists in the half-shells 35a, 35b being elastically deformable, overall or at least in the region of the pockets 41, 43, such that the outer edges of the optical components can be or are clamped in the pockets 41, 43.

This can be achieved for example by the half-shells 35a, 35b being made, entirely or at least in the region of the pockets 41, 43, from a material which is at least to a certain extent elastically deformable, such as a plastic, and/or having a shape in the region of the pockets 41, 43 which enables or effects the clamping of the outer edges of the optical components. The clamping of the optical components in the pockets 41, 43 offers the advantage that any existing manufacturing tolerances are compensated for, and the optical components are fixed in the pockets 41, 43.

A further embodiment consists in the mutually adjacent recesses 37a, 39a, 37b, 39b of each half-shell 35a, 35b being separated from one another by a partition 45 arranged therebetween, such as a partition 45 extending over the entire length of these recesses 37a, 39a, 37b, 39b. The partitions 45 of the half-shells 35a, 35b are preferably designed such that they adjoin one another in pairs when the two half-shells 35a, 35b are arranged on top of one another in such a way that their recesses 37a, 39a, 37b, 39b are opposite one another pairwise.

The partitions 45 offer the advantage that they provide shielding between the signal paths 1, 3 running along the optical axes A1, A2.

Depending on the design of the optical system 100, the half-shells 35a, 35b are, if necessary, designed for example such that two mutually adjacent channels, surrounded by the half-shells 35a, 35b arranged on top of one another and each running parallel to one of the optical axes A1, A2, are connected by a passage opening for a connecting path running from one of the optical axes A1 to the other optical axis A2. This is shown in FIGS. 5 and 6 using the example of a passage opening for the connection path formed here by the useful signal path 11 of the optical system 100 shown in FIG. 1.

In the illustrated exemplary embodiment, the partitions 45 of the half-shells 35a, 35b each have a recess 47a, 47b in the region crossing the connecting path. These recesses 47a, 47b are designed, for example, in such a way that they form the passage opening when the half-shells 35a, 35b are arranged one on top of the other. The passage opening has, for example, a longitudinal axis running coaxially with the connecting path.

Alternatively or additionally, the mount 300 is designed for example such that the half-shells 35a, 35b can be or are mechanically connected to one another.

This connection can be effected for example by the half-shells 35a, 35b being glued to one another, screwed to one another, connected or joined by a joining process such as a welding process, and/or in another way.

Alternatively or additionally, the mount 300 is designed for example such that the half-shells 35a, 35b have mutually complementary plug connector elements 49, 51 by which the half-shells 35a, 35b can be connected or are connected to one another. This offers the advantage that an exact orientation of the two half-shells 35a, 35b to each other is ensured.

FIGS. 5 and 6 show an exemplary embodiment of this in which the half-shells 35a, 35b comprise plug connector elements 49, 51 arranged on the end faces of the partitions 45 of the half-shells 35a, 35b. An embodiment shown in FIGS. 5 and 6 consists in that on the or each partition 45 of one of the two half-shells 35a there is arranged at least one plug connector element 49 designed as a tongue, and the other half-shell 35b comprises for each tongue a plug connector element 51 complementary thereto, designed as a groove. FIGS. 5 and 6 show an exemplary embodiment of this in which the tongue arranged on the partition 45 of the half-shell 35a shown in FIG. 5, and correspondingly on the groove provided in the partition 45 of the other half-shell 35b shown in FIG. 6, each extend over the entire length of the corresponding partition 45.

Connector elements 49, 51 designed as a tongue and groove offer the advantage that by the or each tongue being inserted into the associated groove, high-quality optical shielding extending over the entire length of the partitions 45 is ensured between the channels adjacent to the partitions 45 on both sides, even if the partitions 45 do not lie on top of each other with their entire surface due to manufacturing tolerances.

Alternatively or additionally, the connection of the half-shells 35a, 35b is effected or at least helped, for example, by the mount 300 comprising a sleeve 53 into which the half-shells 35a, 35b arranged one on the other can be or are inserted in such a way that the half-shells 35a, 35b are held together by the sleeve 53. FIG. 7 shows an embodiment in which the mount 300 inserted as an assembly into the housing 23 comprises the sleeve 53 and the two half-shells 35a, 35b arranged one on top of the other in the sleeve 53.

Optionally, the half-shells 35a, 35b are designed for example such that the half-shells 35a, 35b arranged on top of one another can be clamped or are clamped in the sleeve 53. This can be achieved, for example, in that the half-shells 35a, 35b are made entirely or at least in portions from a material that is at least to a certain extent elastically deformable, such as plastic, and/or have a shape that enables and/or effects the clamping of the half-shells 35a, 35b arranged on top of one another in the sleeve 53.

This at least to a certain extent elastic clamping of the half-shells 35a, 35b arranged on top of one another in the sleeve 53 offers the advantage that any existing manufacturing tolerances are compensated for and the optical components arranged in the mount 300 are protected from thermomechanical stresses.

Alternatively, in addition to this, the half-shells 35a, 35b are designed for example such that they have on their outside projecting structures 57, 59, such as projections and/or webs, which are designed and arranged such that, in cooperation with the sleeve 53 surrounding the half-shells 35a, 35b arranged on top of one another, they cause the optical components to be clamped on the outside in the half-shells 35a, 35b.

FIG. 4 shows an embodiment of this in which the structures 57, 59 of each half-shell 35a, 35 each comprise at least one structure 57, 59 for each optical component that can be inserted or is inserted therein, which structure projects outwards relative to a shell region of the corresponding half-shell 35a, 35b, in which the pocket 41, 43 provided for the corresponding optical component is arranged on the inside. In this embodiment variant, the structures 57, 59 are designed for example as elongated structural elements which run in a direction corresponding to the orientation of the optical component which can be or is inserted into the pocket 41, 43.

In this respect, the half-shells 35a, 35b each comprise at least one structure 57 oriented parallel to the corresponding optical axis A1, A2 for each optical component oriented perpendicular to one of the optical axes A1, A2. This is illustrated in FIG. 4 using the example of the structures 57 provided for the clamping of the components of the collimation device 15 shown in FIG. 1, the optical device 17 shown in FIG. 1, the focusing device 19 shown in FIG. 1, and the additional filter 21 shown in FIG. 1, which structures each run parallel to the associated optical axis A1, A2.

Analogously, the half-shells 35a, 35b each comprise, for each component tilted relative to one of the optical axes A1, A2, a structure 59 which is inclined at the angle specified for the corresponding component relative to the corresponding optical axis A1, A2. This is shown in FIG. 4 using the example of the structures 59 provided for the clamping of the filter 5 inclined relative to the first optical axis A1 and the reflector 13 inclined relative to the second optical axis A2, each structure being inclined relative to the corresponding optical axis A1, A2 at the angle provided for the optical component that can be or is inserted therein.

Alternatively, however, the external clamping of the optical components in the half-shells 35a, 35b arranged on top of one another can also be effected in another way. FIG. 8 shows an embodiment of this in which the half-shells 35a, 35a arranged on top of one another in FIG. 8, which are formed in the manner previously described with reference to FIGS. 5 and 6, have openings 61, 63, 65 on the outside adjacent to the channels formed by the recesses 37a, 37b, 39a, 39b.

Here, the openings 61, 63, 65 of each half-shell 35a, 35b are designed and arranged in such a way that at least two openings 61, 63, 65 distributed in the circumferential direction around the outer edge adjoin the outer edge of each optical component inserted into the half-shells 35a, 35b arranged on top of one another, each opening exposing a partial region of the outer edge.

As shown in FIG. 8, the openings 61, 63, 65 distributed around the outer edge of each optical component comprise, for example, at least two openings 61, 63, 65 arranged on opposite sides of the outer edge of the corresponding component, such as an opening 61, 63, 65 arranged in one of the two half-shells 35a and an opening 61, 63, 65 arranged in the other half-shell 35b.

These openings 61, 63, 65 are designed for example as slot-shaped openings each running parallel to one of the optical axes A1, A2. Alternatively or additionally, the openings 61, 63, 65 of each half-shell 35a, 35b comprise, for example, at least one opening 61 each exposing the partial region of the outer edge of a single component, at least one opening 63 each exposing partial regions of the outer edges of at least two optical components, and/or at least one opening 65 extending over the entire length of one of the half-shells 35a, 35b. FIG. 8 shows, as exemplary embodiments of this, the opening 61 which exposes only the partial region of the outer edge of the optical device 17, the opening 63 which exposes the partial region of the outer edge of the collimation device 15 and the partial region of the outer edge of the inclined filter 5, as well as the opening 65 which exposes the partial regions of the outer edges of the reflector 13, the focusing device 19 and the additional filter 21 and extends over the entire length of the corresponding half-shell 35a, 35b.

In the embodiment shown in FIG. 8, the clamping of the optical components in the mount 300 is effected or at least helped in that a molded seal 67, 69, 71 is inserted into each opening 61, 63, 65, and the molded seals 67, 69, 71 are clamped by inserting the half-shells 35a, 35b arranged one on top of the other into the sleeve 53 between the outer edges of the optical components and the sleeve 53 which are adjacent thereto on the inside.

The external clamping of the optical components by the sleeve 53 and the shaped seals 67, 69, 71 offers the advantage that any existing manufacturing tolerances are compensated for by the shaped seals 67, 69, 71, and the optical components are protected from thermomechanical stresses by the shaped seals 67, 69, 71.

1. An optical system for a measuring device for optically measuring a measured variable of a medium, wherein the measured variable can be captured by measurement technology on the basis of useful radiation that results from a wavelength-changing interaction and that is contained in measurement radiation resulting from interactions of excitation radiation with the medium, the optical system comprising:
a first signal path running along a first optical axis; and
a filter inserted into the first signal path, wherein the filter is configured to divide the first signal path into a unidirectional transmission path and a bidirectional path used to transmit the excitation radiation and to receive the measurement radiation,
wherein the filter is designed as a bandpass interference filter,
wherein the filter is inclined relative to the first optical axis such that an inclination angle different from zero degrees is enclosed between a normal vector to the inclined filter and the first optical axis, and
wherein the inclination angle is dimensioned such that the inclined filter:
transmits signal components, lying within a passband of the inclined filter, of the transmission radiation supplied to the unidirectional transmission path as the excitation radiation, and
reflects signal components, lying outside the passband of the inclined filter, of the measurement radiation impinging on the filter via the bidirectional path and resulting from interactions of the transmitted excitation radiation with the medium, and makes them available as useful radiation.

2. The optical system according to claim 1, wherein the inclination angle is greater than or equal to 5° and less than or equal to 45°.

3. The optical system according to claim 2, wherein the inclination angle is dimensioned such that a central wavelength of the inclined filter corresponds to a target wavelength specified for the excitation radiation.

4. The optical system according to claim 1, further comprising:
a collimation device inserted into the transmission path and designed to collimate the transmission radiation fed into the transmission path, wherein the collimation device includes at least one optical component and/or a lens, and/or
an optical device inserted into the bidirectional path and designed to focus the excitation radiation emerging from the optical system via the bidirectional path onto a region lying outside the optical system and to collimate the measurement radiation entering the bidirectional path from the region, wherein the optical device includes at least one optical component, a lens, and/or an objective.

5. The optical system according to claim 1, further comprising:
a second signal path running along a second optical axis or a second optical axis running parallel to the first optical axis;
a reflector inserted into the second signal path at the end,
wherein the reflector is arranged relative to the inclined filter and is inclined relative to the second optical axis in such a way that the useful radiation reflected by the inclined filter impinges on the reflector and is reflected in a direction parallel to the second optical axis.

6. The optical system according to claim 5, further comprising:
a focusing device inserted into the second signal path and designed to focus the useful radiation emerging from the optical system via the second signal path onto a region located inside or outside the optical system, wherein the focusing device includes at least one optical component and/or a lens, and/or
an additional filter inserted into the second signal path and configured to limit a wavelength range of the useful radiation transmitted through the additional filter to a measuring wavelength range specified depending on the measured variable to be measured.

7. A mount for an optical system having at least one optical component arranged in a first signal path running along a first optical axis and at least one optical component arranged in a second signal path running along a second optical axis or along a second optical axis running parallel to the first optical axis, the mount comprising:
two half-shells,
wherein each half-shell has for each optical axis a recess running parallel to the optical axis,
wherein each half-shell can be positioned or is positioned on top of the other half-shell in such a way that each pair of recesses of the half-shells arranged on top of one another, formed by two opposite, adjacent recesses, forms in each case a channel whose longitudinal axis is coaxial with one of the optical axes,
wherein each half-shell for each optical component of the optical system has a pocket adjacent to one of the recesses into which a partial region of an outer edge of the corresponding optical component can be or is inserted, and
wherein each pocket is arranged at a position provided for the corresponding optical component that can be inserted or is inserted therein within the optical system in an orientation specified for the optical component relative to the optical axis running through the optical component in the optical system.

8. The mount according to claim 7,
wherein the adjacent recesses of each half-shell are separated from each other by a partition arranged therebetween,
wherein the half-shells are designed such that:
the partitions of the half-shells arranged on top of one another are adjacent to each other,
the partitions extend over the entire length of the recesses adjacent thereto, and/or
in each of the partitions of the half-shells a recess is provided that is designed such that the mutually adjacent recesses of the half-shells arranged on top of one another form a passage opening for a connecting path running from one of the optical axes to the other optical axis, and/or
wherein the half-shells can be connected or are connected to one another by mutually complementary plug connector elements arranged on the end faces of the partitions of the half-shells.

9. The mount according to claim 8,
wherein the adjacent recesses of each half-shell are separated from each other by the partition arranged therebetween, and
wherein at least one plug connector element designed as a tongue or as a tongue running over the entire length of the partition is arranged on the partition of at least one of the two half-shells, and the partition of the other half-shell includes, for each plug connector element designed as a tongue, a plug connector element complementary thereto designed as a groove.

10. The mount according to claim 9, further comprising:
a sleeve,
wherein the half-shells arranged on top of one another can be inserted or are inserted into the sleeve, and/or
wherein the half-shells are designed such that the half-shells arranged on top of one another can be inserted or are inserted into the sleeve, and/or they consist entirely or at least in portions of an elastically deformable material or an elastically deformable plastic and/or have a shape which enables and/or effects the clamping of the half-shells arranged on top of one another in the sleeve.

11. The mount according to claim 10,
wherein the half-shells have on their outer side protruding structures and/or structures designed as projections as connecting elements or as elongated structural elements that are designed and arranged such that, in cooperation with the sleeve surrounding the half-shells arranged on top of one another, they effect an external clamping of the optical components arranged in the half-shells arranged on top of one another.

12. The mount according to claim 11,
wherein the structures of each half-shell include for each optical component at least one structure which projects outwards relative to a shell region of the corresponding half-shell in which the pocket provided for the optical component is arranged on the inside, and/or
wherein the half-shells for at least one or each optical component oriented perpendicular to one of the optical axes in the optical system each includes at least one structure running parallel to the optical axis, and/or
wherein the half-shells for at least one or each optical component tilted in the optical system relative to one of the optical axes by an angle specified for the corresponding component each comprise a structure running at the angle specified for the component relative to the corresponding optical axis.

13. The mount according to claim 10,
wherein the half-shells have openings into each of which a shaped seal can be or is inserted,
wherein the openings of each half-shell are designed and arranged in such a way that at least two openings distributed in the circumferential direction around the outer edge adjoin the outer edge of each optical component inserted into the half-shells arranged on top of one another, each opening exposing a partial region of the outer edge of the optical component, and
wherein the half-shells arranged on top of one another, with the optical components inserted therein and the shaped seals inserted into the openings, can be inserted or are inserted into the sleeve in such a way that the shaped seals are clamped between the partial regions, adjacent thereto on the inside, of the outer edges of the optical components and the sleeve.

14. The mount according to claim 13,
wherein the half-shells are elastically deformable at least in the region of the pockets such that the optical components can be or are clamped in the pockets,
wherein the half-shells as a whole or at least in the region of the pockets consist of an elastically deformable material or an elastically deformable plastic and/or in the region of the pockets have a shape that enables and/or effects the clamping of the optical components, and/or
wherein the half-shells include mutually complementary plug connector elements by which the half-shells can be or are mechanically connected to one another.

15. A measuring device for measuring a measured variable of a medium with an optical system according to claim 6, the measuring device comprising:
a housing in which the optical system is arranged;
a transmitting device with a radiation source which is designed to feed the transmitted radiation generated by the radiation source into the transmission path of the optical system; and
a detection device which is designed to receive the useful radiation emitted by the optical system and to determine and provide at least one property of the useful radiation which is dependent on the measured variable.

16. The measuring device according to claim 15, further comprising:
a mount, including:
two half-shells,
wherein each half-shell has for each optical axis a recess running parallel to the optical axis,
wherein each half-shell can be positioned or is positioned on top of the other half-shell in such a way that each pair of recesses of the half-shells arranged on top of one another, formed by two opposite, adjacent recesses, forms in each case a channel whose longitudinal axis is coaxial with one of the optical axes,
wherein each half-shell for each optical component of the optical system has a pocket adjacent to one of the recesses into which a partial region of an outer edge of the corresponding optical component can be or is inserted, and
wherein each pocket is arranged at a position provided for the corresponding optical component that can be inserted or is inserted therein within the optical system in an orientation specified for the optical component relative to the optical axis running through the optical component in the optical system.

17. The measuring device according to claim 16, further comprising:
a transparent process interface through which the measuring device transmits excitation radiation into the medium and receives measurement radiation resulting from interactions of the transmitted excitation radiation with the medium; and
an evaluation device which is connectable or connected to the detection device and which is designed to determine and provide measurement results of the measured variable based on the property or properties of the useful radiation determined by the detection device,
wherein the measuring device is designed as a fluorescence measuring device or as a Raman spectrometric measuring device, and/or
wherein the measuring device is designed as a fluorescence measuring device in such a way that:
the radiation source is designed to generate transmitted radiation in a wavelength range matched to a fluorescent component of the medium and/or in a wavelength range of 180 nm to 1200 nm, and/or
the inclination angle is dimensioned such that the inclined filter has a central wavelength which corresponds to a desired wavelength with which a fluorescent component of the medium can be excited to fluorescence.
